(19) United States
(12) Patent Application Publication
(10) Pub. No.: US 2014/0330168 A1
(43) Pub. Date: Nov. 6, 2014

(54) MULTIFUNCTION STETHOSCOPE

(71) Applicant: Angela Beeson, South Ozone Park, NY (US)

(72) Inventor: Angela Beeson, South Ozone Park, NY (US)

(21) Appl. No.: 13/875,545

(22) Filed: May 2, 2013

(51) Int. Cl. A61B 5/00; A61B 19/00; A61B 7/02

(52) U.S. Cl. CPC A61B 5/7445; A61B 7/02; A61B 19/5202
USPC 600/586

(57) ABSTRACT

A multifunction stethoscope comprising a stethoscope, where the stethoscope includes a tubing and a headset; a chest piece attached to an end of the tubing, where the chest piece includes a diaphragm and a bell; a time display positioned on the chest piece, where the time display includes a stopwatch; and a light positioned on the chest piece, where the light includes an on/off button for activation. The multifunction stethoscope allows a user to utilize a light and timer with the standard functions of the stethoscope.

### BACKGROUND OF THE INVENTION

1. Field of the Invention

The present invention relates to a stethoscope with accessories added to the chest piece. The accessories include helpful devices such as a stopwatch and a penlight.

2. Description of Related Art

Stethoscopes are tools used by medical professionals as a way to listen to the internal sounds of a human or animal body, and are often worn by doctors and nurses as a symbol of their profession. The usual sounds heard with the stethoscope are heart sounds, movement within the lungs and intestines, and blood flow through veins and arteries. Most often, the doctor or nurse uses the stethoscope when taking a patient's vitals during rounds. They listen for the pulse, time the heartbeats and note their findings in the patient's chart. Typically, the doctor or nurse is struggling to hold the stethoscope, timer and pen all at once to record an accurate reading. Also, during night rounds the patient room is often dark making notations difficult to write and read.

Recently, some stethoscopes have been developed with added features to make the stethoscopes more multi-purpose tools. For example, some stethoscopes include timers or stopwatches attached to the tubing to alleviate having to carry a separate watch or timer while checking vitals. But these timers are bulky and may cause excessive weight and strain around the wearer's neck after an extended period of time. Also, U.S. Pat. No. 5,638,453 by Bruce E. McLaughlin discloses a transducer enhanced stethoscope which includes a temperature gauge on the chest piece of the stethoscope. This way the nurse is able to check the patient's temperature while they listen to their pulse. Finally, U.S. Pat. No. 7,527,123 by Mark Puder discloses a patient-friendly stethoscope which includes an optical fiber within the tubing to illuminate the chest piece, the tubing, the rim of the diaphragm and the bell. While the optical fiber illuminates majority of the stethoscope, it is meant for entertainment purposes and is not designed to cast a concentrated light for reading or writing.

While these devices do provide added convenience and novelty to the currently available stethoscopes, it would still be desirable in the art to provide a stethoscope that includes a timer that is not bulky nor adds additional weight. It would also be desirable in the art to provide a stethoscope with concentrated lighting to allow a user to see what they are writing in little to no light environments.

### SUMMARY OF THE INVENTION

In view of the foregoing disadvantages inherent in the prior art, the general purpose of the present invention is to provide a stethoscope with additional functions, configured to include all of the advantages of the prior art, and to overcome the drawbacks inherent therein.

Accordingly, an object of the present invention is to provide a stethoscope with a timer display on the chest piece; positioned either on the diaphragm or the bell.

Another object of the invention is to provide a stethoscope with a light positioned on the chest piece, thereby allowing a user to write in dim or dark rooms.

To achieve the above objects, in an aspect of the present invention, a multifunction stethoscope is described comprising a stethoscope, where the stethoscope includes a tubing and a headset; a chest piece attached to an end of the tubing, where the chest piece includes a diaphragm and a bell; a time display positioned on the chest piece, where the time display includes a stopwatch; and a light positioned on the chest piece, where the light includes an on/off button for activation. The multifunction stethoscope allows a user to utilize a light and timer with the standard functions of the stethoscope.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing in which:

FIG. 1 depicts a perspective view of a multifunction stethoscope in accordance with an exemplary embodiment of the present invention.

### DETAILED DESCRIPTION OF THE DRAWINGS

The present invention relates to a stethoscope with accessories added to the chest piece. The accessories include helpful devices such as a stopwatch and a penlight. The present invention provides a multifunction stethoscope to simultaneously listen to a body sounds, time heartbeats and illuminate the writing area all with a single device. By utilizing all three functions in a solitary tool, doctors, nurses and other health professionals are able to quickly, easily and unobtrusively check a patient's vitals.

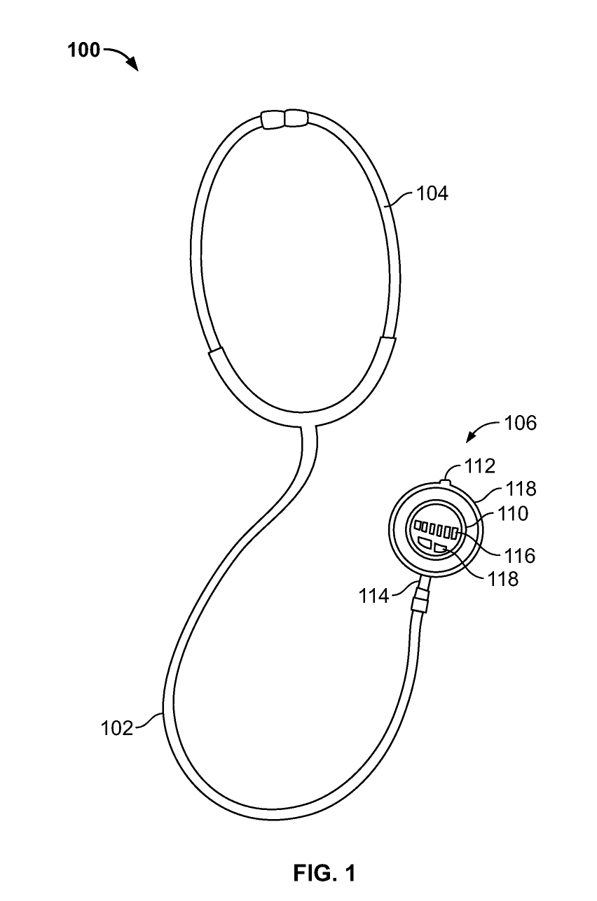

Turning now descriptively to the drawings, referring to FIG. 1, a perspective view of a multifunction stethoscope 100 is shown in accordance with an exemplary embodiment of the present invention. The multifunction stethoscope 100 includes a tubing 102 connected to a headset 104. At an end of the tubing 102 is a chest piece 106. The chest piece 106 is primarily made up of a diaphragm 108 and a bell 110. The diaphragm 108 and the bell 110 are the portions of the multifunction stethoscope 100 that allow a user to hear the internal and superficial acoustics of the human body. For example, the diaphragm 108 enables a user to listen to the internal sounds like a heart sounds, and lungs. The bell 110 allows the user to hear external sounds like vibrations of the user's skin. The sound vibrations travel through the tubing 102 and into the headset 104 to allow the healthcare worker to carefully listen to the body sounds.

Positioned at an edge of the diaphragm 108 may be a light 112. The light 112 may be a small LED light to illuminate a writing surface when a user is working in dark or dim area. The light 112 may also be used to illuminate a patient's pupils, body cavities and fluid filled areas. An on/off button 114 may be positioned at the opposite end of the diaphragm 108 to turn the light 112 on and off as needed. Alternatively, the light 112 and on/off button 114 may be attached to the bell 110 depending on the stylizing preferred by the user.

Atop the bell 110 may be a time display 116 to display the time in hours, minutes and seconds. The time display 116 may also function as a stopwatch allowing the user to time the body sounds while they are checking the patient's vitals. A pair of start/reset buttons 118 may be positioned above or below the time display 116 to allow the user from switch between displaying the current time and the timer of the stopwatch. The time display 116 may alternatively be positioned on the diaphragm 108. While using the time display 116 the internal workings of the timer do not interfere with the sounds accumulated in the chest piece. This way the time display 116 may be placed on either the diaphragm 108 or the bell 110 without interrupting the sounds heard in the headset 104. Also, the time display 116 does not add any additional weigh to the chest piece 106, so that the multifunction stethoscope 100 is still comfortable for the user to wear.

With the multifunction stethoscope the user is afforded the convenience of a time display and a light included with the standard functions of the stethoscope. The user does not have to hassle with scrambling to hold the diaphragm and timer, and then to move to a separate location to see the notes they are taking in the patient's chart. All of the functions may be done at once by using the useful tools associated with the multifunction stethoscope.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A multifunction stethoscope comprising:
a. a stethoscope, where the stethoscope includes a tubing and a headset;
b. a chest piece attached to an end of the tubing, where the chest piece includes a diaphragm and a bell;
c. a time display positioned on the chest piece, where the time display includes a stopwatch; and
d. a light positioned on the chest piece, where the light includes an on/off button for activation.

2. The multifunction stethoscope according to claim 1, where the time display includes a pair of start/reset buttons.

3. The multifunction stethoscope according to claim 1, where the time display is positioned on the bell.

4. The multifunction stethoscope according to claim 1, where the time display is positioned on the diaphragm.

5. The multifunction stethoscope according to claim 1, where the light is positioned on an edge of the diaphragm.

6. The multifunction stethoscope according to claim 1, where the light is positioned on the bell.

7. The multifunction stethoscope according to claim 1, where the light is an LED light.
